(19) United States
(12) Patent Application Publication
(10) Pub. No.: US 2024/0307683 A1
(43) Pub. Date: Sep. 19, 2024

(54) SKIN INFLAMMATION TREATMENT DEVICE

(71) Applicant: Proxihealthcare Inc., Ulsan (KR)

(72) Inventor: Young Wook Kim, Seoul (KR)

(21) Appl. No.: 18/510,666

(22) Filed: Nov. 16, 2023

Related U.S. Application Data
(63) Continuation of application No. PCT/KR2021/019087, filed on Dec. 15, 2021.

(30) Foreign Application Priority Data
May 18, 2021 (KR) 10-2021-0064417

(51) Int. Cl. A61N 1/20; A61N 1/32

(52) U.S. Cl. CPC A61N 1/205; A61N 1/328

(57) ABSTRACT

A skin inflammation treatment device includes: a case; one or more electrodes which are located in the case and which receive a driving signal to provide the electromagnetic wave corresponding to the driving signal; and a signal supply, which is located in the case, generates the driving signal by mixing an AC signal with a DC signal and supplies the driving signal to the electrodes.

### CROSS REFERENCE TO RELATED APPLICATIONS

This application is a Bypass Continuation of International Application No. PCT/KR2021/019087, filed on Dec. 15, 2021, and claims priority from and the benefit of Korean Patent Application No. 10-2021-0064417, filed on May 18, 2021, each of which is incorporated by reference for all purposes as if fully set forth herein.

### BACKGROUND

### Field

The present invention relates to a device for treating skin inflammation using special microcurrent electromagnetic waves.

### Discussion of the Background

In general, devices for treating skin employ a method of transmitting ultrasonic waves to skin tissue (HIFU type), a method of transmitting electromagnetic waves to skin tissue (specially, high frequency and RF type), a method of irradiating laser light to skin tissue (optical type), and the like. A method of using plasma to heal skin wounds or to promote the treatment process of skin diseases such as atopy has also been used.

These skin treatment devices remove damaged collagen, elastic fibers, and the like in the deep layers of the skin to promote new formation, and exhibits effects of improving skin conditions such as pigmentation, acne scars, and wrinkles.

Recently, the demand for various skin treatment devices for acne removal, wrinkle removal, skin elasticity restoration, sebum removal, and the like has increased, so there is a need to develop effective skin treatment devices, especially for removing wastes and relieving inflammation, which are the fundamental problems of skin treatment.

The above information disclosed in this Background section is only for understanding of the background of the inventive concepts, and, therefore, it may contain information that does not constitute prior art.

### SUMMARY

An object of the present invention for solving the above-mentioned problems is to provide a skin inflammation treatment device that may be expected to have a therapeutic effect on skin inflammation by providing electromagnetic waves.

In addition, another object of the present invention is to provide a skin inflammation treatment device that amplifies an inflammation management effect by using a driving signal generated by mixing AC and DC signals.

Additional features of the inventive concepts will be set forth in the description which follows, and in part will be apparent from the description, or may be learned by practice of the inventive concepts.

According to one aspect of the invention, a skin inflammation treatment device includes: a case; at least one electrode located in the case and configured to receive a driving signal and provide electromagnetic waves corresponding to the driving signal; and a signal supply located in the case and configured to generate the driving signal by mixing an AC signal and a DC signal, and to supply the driving signal to the electrode.

The case may include: a first case having an opening that exposes at least a portion of the electrode; and a second case provided with a switch that controls the operation of the skin inflammation treatment device.

The first case may include a protrusion region, and the opening may be formed in the protrusion region.

The skin inflammation treatment device may further include: a first circuit board on which the electrode is mounted on a front surface thereof; a second circuit board connected to the first circuit board through a connection line and on which the signal supply is mounted; and a battery configured to supply a battery voltage to the signal supply.

The electrode may include a first electrode and a second electrode arranged to be spaced apart from each other, wherein the first electrode may include a first insertion protrusion formed to protrude from one surface of the first electrode, the second electrode may include a second insertion protrusion formed to protrude from one surface of the second electrode, and the first circuit board may include a first receiving groove configured to receive the first insertion protrusion and a second receiving groove configured to receive the second insertion protrusion.

The first circuit board may include: a first connection terminal located on a rear surface of the first circuit board and electrically connected to the first electrode; and a second connection terminal located on the rear surface and electrically connected to the second electrode.

The skin inflammation treatment device may further include: a first indicator provided on the second case and configured to indicate a charging status of the battery; and a second indicator provided on the second case and configured to indicate an operation time of the skin inflammation treatment device.

The skin inflammation treatment device may further include a controller configured to control the signal supply to change at least one of characteristics of the driving signal under a user's control.

The characteristics of the driving signal may include amplitude and DC offset.

According to the present invention, it is possible to provide a skin inflammation treatment device that can be expected to have a therapeutic effect on skin inflammation by providing electromagnetic waves.

In addition, according to the present invention, it is possible to provide a skin inflammation treatment device that amplifies an inflammation management effect by using a driving signal generated by mixing AC and DC signals.

It is to be understood that both the foregoing general description and the following detailed description are illustrative and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate illustrative embodiments of the invention, and together with the description serve to explain the inventive concepts.

FIG. 1 is a diagram schematically illustrating a configuration of a skin inflammation treatment device according to an embodiment of the present invention.

FIGS. 2A and 2B are diagrams illustrating a biofilm removal effect of a driving signal generated by mixing AC and DC signals.

FIGS. 3 and 4 are diagrams illustrating a skin inflammation treatment device according to an embodiment of the present invention in a specific form.

FIG. 5 is a diagram illustrating an internal configuration of a skin inflammation treatment device according to an embodiment of the present invention in a specific form.

FIG. 6A is a diagram illustrating a first electrode and a second electrode.

FIG. 6B is a diagram illustrating a first circuit board shown in FIG. 5.

FIG. 7 is diagram illustrating an internal configuration of a signal supply according to an embodiment of the present invention.

FIGS. 8A, 8B, and 8C are diagrams illustrating waveforms of signals according to an embodiment of the present invention.

FIG. 9 is a diagram illustrating an internal configuration of a skin inflammation treatment device according to another embodiment of the present invention.

FIGS. 10A, 10B, 10C, and 10D are diagrams illustrating the shape and arrangement of electrodes according to another embodiment of the present invention.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing illustrative features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

In addition, it should be clarified that the division of the elements herein is merely based on the primary function performed by each element. That is, two or more elements to be described below may be combined into a single element, or a single element may be divided into two or more elements for more detailed functions. In addition to its primary function, each element to be described below may additionally perform some or all of the functions performed by other elements, and a portion of the primary function of each element may be exclusively performed by other elements.

Hereinafter, a device for treating skin inflammation according to an embodiment of the present invention will be described with reference to the drawings related to embodiments of the present invention.

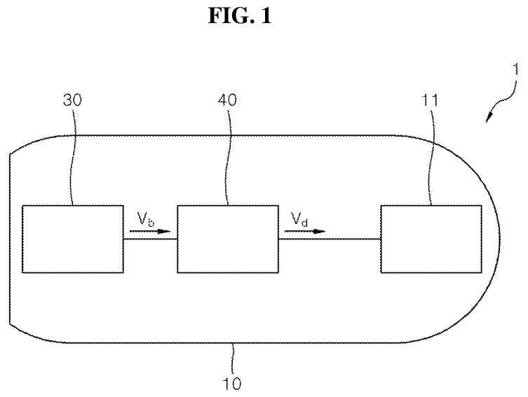

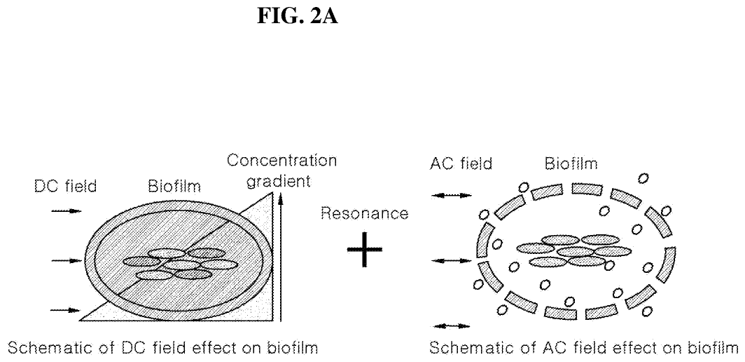

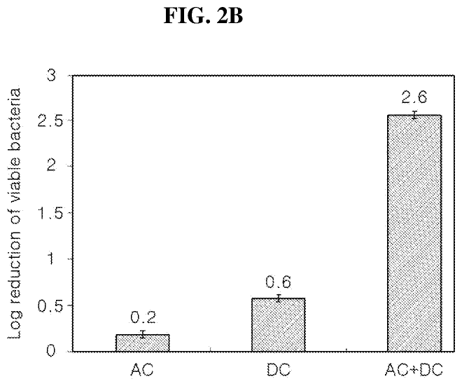

FIG. 1 is a diagram schematically illustrating a configuration of a skin inflammation treatment device according to an embodiment of the present invention; and FIGS. 2a and 2b are diagrams illustrating a biofilm removal effect of a driving signal generated by mixing AC and DC signals.

Referring to FIG. 1, a skin inflammation treatment device 1 according to an embodiment of the present invention may include a case 10, an electrode 11, a battery 30, and a signal supply 40. The electrode 11, the battery 30, and the signal supply 40 may be provided in the case 10.

The electrode 11 may receive a driving signal Vd from the signal supply 40 and generate an electric field corresponding to the driving signal Vd.

The electrode 11 may provide electromagnetic waves based on the electrical energy of the driving signal Vd, and these electromagnetic waves may electrically affect a user's skin to provide effects such as inflammation relief through biofilm removal. In addition, the electrode 11 may enhance the effect of injecting drugs (e.g., acne treatment ointment, moisturizing ointment, microcurrent amplification ointment, and the like) by electrically affecting the user's skin.

When a plurality of electrodes 11 are provided in the skin inflammation treatment device 1, the driving signal Vd may be applied to at least one of the electrodes 11, and an electrode that serves as a ground electrode may be additionally installed on the skin inflammation treatment device 1.

The electrode 11 may be formed of a material such as brass, aluminum, conducting polymer, conducting silicon, or stainless steel, but is not limited thereto, and any material with conductivity may be used as the electrode material.

The battery 30 may be set as a primary battery or a secondary battery.

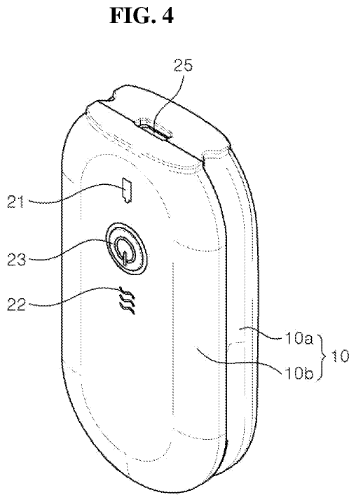

If the battery 30 is a primary battery, the battery 30 may be replaced periodically by the user, and if the battery 30 is a secondary battery, charging may be performed by various charging methods. For example, charging may be performed using a wired charging method through a charging terminal 25 (see FIG. 4) to be described later, or using a wireless charging method.

The signal supply 40 may generate the driving signal Vd by using a battery voltage Vb supplied from the battery 30.

In particular, the signal supply 40 generates the driving signal Vd by mixing an alternating current (AC) signal and a direct current (DC) signal.

Accordingly, the driving signal Vd may contain both AC and DC components, and synergistic effects and resonance may occur due to simultaneous application of the AC and DC components, thereby enhancing the removal effect of the biofilm that causes inflammation.

Referring to FIG. 2A, an electric field induced by the DC component may increase the structural stress of the biofilm by inducing an imbalance in the local distribution of charges, while an electric field induced by the AC component may increase the permeability of the external protector through the generation of specific vibrations.

The synergistic effect between these AC and DC components may be observed in FIG. 2B. Specifically, it can be seen that the biofilm removal effect is significantly superior when the electric fields induced by the AC component and the DC voltage are provided simultaneously in an overlapping manner, compared to when the electric fields induced by the AC component and the DC voltage are provided separately.

Depending on the driving signal Vd supplied from the signal supply 40 according to an embodiment of the present invention, the electric field induced by the DC component and the electric field induced by the AC component may be provided simultaneously from the electrode 11, so that the amplified removal effect on the biofilm described above may be achieved.

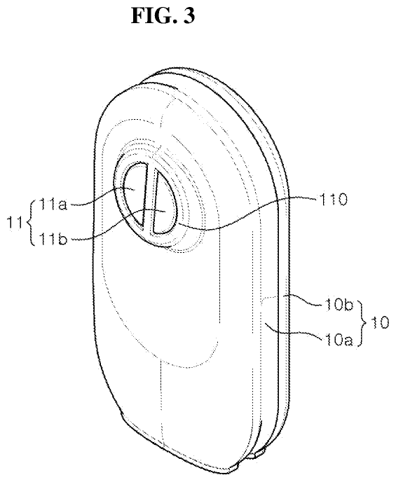

FIGS. 3 and 4 are diagrams illustrating a skin inflammation treatment device according to an embodiment of the present invention in a specific form. In particular, FIG. 3 depicts a perspective view of a first surface of the skin inflammation treatment device 1 or the case 10 forming the body of the skin inflammation treatment device 1, while FIG. 4 shows a perspective view of a second surface of the skin inflammation treatment device 1 or the case 10. Here, the second surface may be an opposite surface of the first surface.

Referring to FIGS. 3 and 4, the case 10 may include a first case 10a and a second case 10b. The first case 10a and the second case 10b may be combined to form the entire case 10.

The first surface (front or top surface) of the case 10 may be included in the first case 10a, and the second surface (rear or bottom surface) of the case 10 may be included in the second case 10b.

As shown in FIG. 3, the electrode 11 may be disposed in the case 10, and the electrode 11 may include a first electrode 11a and a second electrode 11b.

The first electrode 11a and the second electrode 11b may be disposed on the first surface of the case 10 while being spaced apart from each other, and more specifically may be disposed on the first case 10a. The first case 10a may be provided with an opening that allows a portion of the first and second electrodes 11a and 11b to be exposed.

The first electrode 11a and the second electrode 11b may provide electromagnetic waves by the driving signal Vd supplied from the signal supply 40. For example, the first electrode 11a may be set as a positive electrode, and the second electrode 11b may be set as a negative electrode or a ground electrode.

Meanwhile, in the first case 10a, a protrusion region 110 may be formed, protruding from at least a portion of the first surface of the first case 10a toward the outside of the first case 10a, and the first electrode 11a and the second electrode 11b may be positioned in the protrusion region 110. That is, the opening formed in the first case 10a to expose the first electrode 11a and the second electrode 11b may be provided in the protrusion region 110.

With the protrusion region 110 formed in the first case 10a, the first electrode 11a and the second electrode 11b located in the protrusion region 110 may be easily brought into contact with the user's skin when using the skin inflammation treatment device 1.

Referring to FIG. 4, the skin inflammation treatment device 1 according to an embodiment of the present invention may include a first indicator 21, a second indicator 22, a switch 23, and the charging terminal 25.

The first indicator 21, the second indicator 22, and the switch 23 may be disposed on the second surface of the case 10, and more specifically, on the second case 10b.

The first indicator 21 may indicate the charging status of the battery disposed within the skin inflammation treatment device 1. For example, the first indicator 21 may be provided with light emitting diodes (LED) capable of emitting light in multiple colors, and may indicate battery states such as charging (e.g., in red) and fully charged (e.g., in blue).

The second indicator 22 may indicate the operation time of the skin inflammation treatment device 1. For example, the second indicator 22 may be provided with LEDs capable of emitting light in a plurality of colors, and may indicate the operation time such as 15 minutes (e.g., in green) or 30 minutes (e.g., in yellow).

The switch 23 may be used to control the power on/off of the skin inflammation treatment device 1 and to set the operation time.

The charging terminal 25 may be located on the side surface of the case 10 and may be used to charge the battery 30 disposed inside the skin inflammation treatment device 1.

However, the present invention is not limited thereto, and when a replaceable battery is inserted into the skin inflammation treatment device 1, the charging terminal 25 may be omitted. In this case, the first indicator 21 may indicate the remaining lifespan status or the like of the battery. In addition, when a replaceable battery is inserted, the first case 10a and the second case 10b may be designed to be capable of being coupled to and detached from each other.

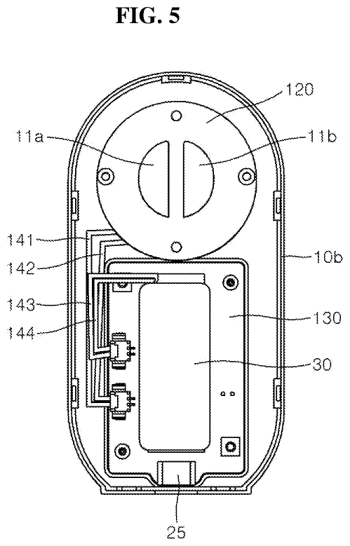

FIG. 5 is a diagram illustrating an internal configuration of the skin inflammation treatment device according to an embodiment of the present invention in a specific form. In particular, FIG. 5 shows the skin inflammation treatment device 1 shown in FIGS. 3 and 4 with the first case 10a removed.

Referring to FIG. 5, the first electrode 11a, the second electrode 11b, a first circuit board 120, a second circuit board 130, and the battery 30 may be accommodated in the case 10.

The first case 10a and the second case 10b may provide an area and space for installing components such as the first circuit board 120 and the second circuit board 130.

For example, a receiving portion in which the first circuit board 120 can be seated and fixed may be formed in the first case 10a, and a receiving portion in which the second circuit board 130 can be seated and fixed may be formed in the second case 10b. The first circuit board 120 and the second circuit board 130 may be fixed to the first case 10a and the second case 10b, respectively, through a fastening member (not shown).

The first electrode 11a and the second electrode 11b may be mounted on the first circuit board 120.

The signal supply 40 that generates the driving signal Vd may be mounted on the second circuit board 130, and a controller 50 may be further mounted thereon.

In addition, a charging circuit for charging the battery 30 using an externally supplied current may be mounted on the second circuit board 130.

A first connection line 141 and a second connection line 142 may be connected to the second circuit board 130, and may supply the driving signal Vd to the first electrode 11a and the second electrode 11b. To this end, the first connection line 141 may be electrically connected to the first electrode 11a and the second connection line 142 may be electrically connected to the second electrode 11b.

A third connection line 143 and a fourth connection line 144 may be connected to the second circuit board 130, and may connect the charging circuit mounted on the second circuit board 130 to the battery 30.

Meanwhile, although the skin inflammation treatment device 1 is shown in FIG. 5 as having the first circuit board 120 and the second circuit board 130, the present invention is not limited to this, and the electrode 11, the signal supply 40, the charging circuit, the controller 50, and the like may be mounted on one circuit board.

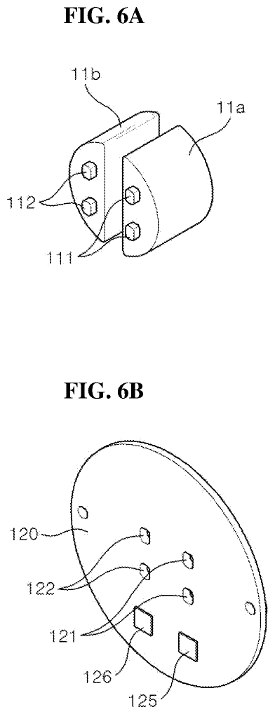

FIG. 6A is a diagram illustrating a first electrode and a second electrode, and FIG. 6B is a diagram illustrating a first circuit board shown in FIG. 5. In particular, FIG. 6B shows the rear surface of the first circuit board 120, i.e., a surface opposite to the front surface where the first electrode 11a and the second electrode 11b are seated.

Referring to FIG. 6A, the first electrode 11a may include at least one first insertion protrusion 111. The first insertion protrusion 111 may be formed to protrude from one surface of the first electrode 11a, and may be inserted into a first receiving groove 121 provided in the first circuit board 120 when the first electrode 11a is mounted on the first circuit board 120. As the first insertion protrusion 111 is inserted into the first receiving groove 121 provided in the first circuit board 120, the first electrode 11a may be fixed to the first circuit board 120.

The second electrode 11b may include at least one second insertion protrusion 112, and the second insertion protrusion 112 may be formed to protrude from one surface of the second electrode 11b. When the second electrode 11b is mounted on the first circuit board 120, the second insertion protrusion 112 may be inserted into a second receiving groove 122 provided in the first circuit board 120, thereby allowing the second electrode 11b to be fixed to the first circuit board 120.

For simplicity of manufacturing, the first electrode 11a and the first insertion protrusion 111 may be integrally formed, and the second electrode 11b and the second insertion protrusion 112 may be integrally formed.

Meanwhile, FIG. 6A shows two insertion protrusions formed in each of the electrodes, but the present invention is not limited thereto, and the shape and/or number of insertion protrusions formed in each of the electrodes may be varied.

Referring to FIG. 6B, the first circuit board 120 may be provided with at least one first receiving groove 121, at least one second receiving groove 122, a first connection terminal 125, and a second connection terminal 126.

The first receiving groove 121 may receive the first insertion protrusion 111, and, for this purpose, may be formed to correspond to the number and shape of the first insertion protrusion 111.

The second receiving groove 122 may receive the second insertion protrusion 112, and, for this purpose, may be formed to correspond to the number and shape of the second insertion protrusion 112.

The first connection line 141 and the second connection line 142 may be connected to the first connection terminal 125 and the second connection terminal 126, respectively.

The first connection terminal 125 may transmit the driving signal Vd or ground voltage transmitted from the first connection line 141 to the first electrode 11a through the first circuit board 120, and the second connection terminal 126 may transmit the driving signal Vd or ground voltage transmitted from the second connection line 142 to the second electrode 11b through the first circuit board 120.

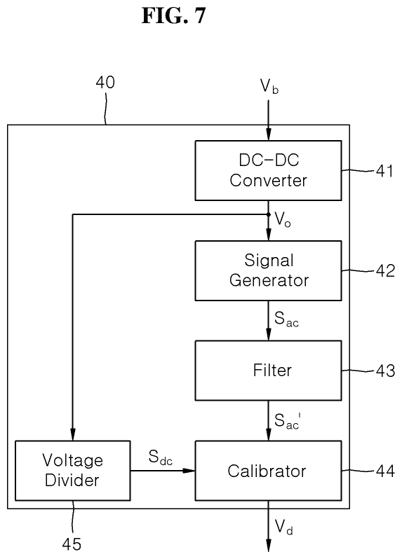

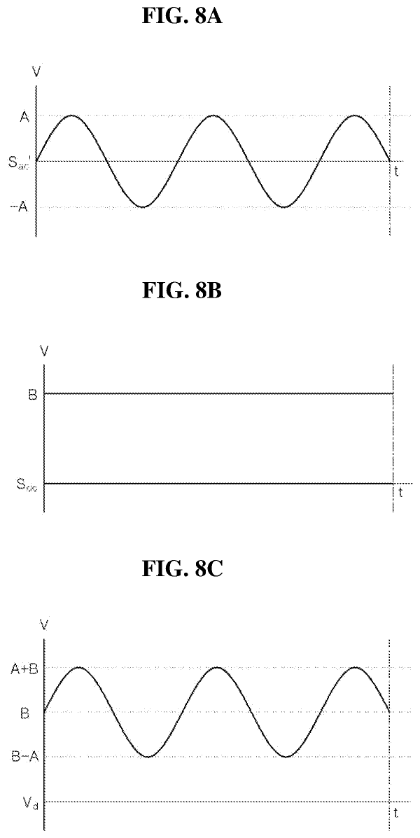

FIG. 7 is a diagram illustrating an internal configuration of a signal supply according to an embodiment of the present invention. FIGS. 8A to 8C are diagrams illustrating waveforms of signals according to an embodiment of the present invention. In particular, FIG. 8A shows a filtered AC signal Sac′, FIG. 8B shows a DC signal Sdc, and FIG. 8C shows the driving signal Vd generated by mixing the filtered AC signal Sac′ and DC signal Sdc.

Referring to FIG. 7, the signal supply 40 according to an embodiment of the present invention may include a DC-DC converter 41, a signal generator 42, a filter 43, and a calibrator 44, and may further include a voltage divider 45.

The DC-DC converter 41 may receive the battery voltage Vb from the battery 30, and may convert the battery voltage Vb into a predetermined level of output voltage Vo to output it.

The signal generator 42 may operate based on the voltage supplied from the DC-DC converter 41, and may generate an AC signal Sac with a predetermined frequency using the output voltage Vo of the DC-DC converter 41.

The signal generator 42 may be implemented using a known configuration, such as an oscillator or a function generator, capable of generating an AC signal.

For example, the AC signal Sac may be set to a frequency of 1 KHz to 1000 MHz. This is because if the AC signal Sac is set to a low frequency of less than 1 KHz, the effect such as alleviating inflammation through biofilm removal is reduced, and if the AC signal Sac is set to an ultra-high frequency of more than 1000 MHz, the effect of biofilm removal is also reduced. Meanwhile, the AC signal Sac may be set to a frequency of 5 MHz to 15 MHZ, which is suitable for biofilm removal.

In addition, the amplitude of the AC signal Sac may be set to 0.1 mV to 3V. This is because if the amplitude of the AC signal Sac is less than 0.1 mV, it is difficult to expect the biofilm removal effect, and if the amplitude of the AC signal Sac exceeds 3V, there is a risk that toxic substances may be generated due to electrolysis of body fluids.

The filter 43 may perform a filtering operation on the AC signal Sac generated by the signal generator 42. For example, the filter 43 may include a low pass filter and may convert the AC signal Sac in the form of a sawtooth wave into the AC signal Sac′ in the form of a sine wave. However, the type of the filter 43 is not limited thereto, and various types of filters may be employed depending on the design structure.

The calibrator 44 may generate the driving signal Vd by mixing the DC signal Sdc with the AC signal Sac′ supplied through the filter 43. For example, the calibrator 44 may be implemented with an operating amplifier capable of summing (or superimposing) the AC signal Sac′ and the DC signal Sdc, but is not limited thereto.

Accordingly, an offset corresponding to the DC signal Sdc may be generated in the AC signal Sac′, and the driving signal Vd containing both AC and DC components may be generated.

Since the driving signal Vd contains all the characteristics of the AC signal Sac, the driving signal Vd may be set to a frequency of 1 KHz to 1000 MHz, and further, may be set to a frequency of 5 MHz to 15 MHz, which is more suitable for biofilm removal. In addition, the amplitude of the driving signal Vd may be set to 0.1 mV to 3V.

Referring to FIG. 8A, the calibrator 44 may receive the AC signal Sac′ having an amplitude of A volt (V) from the filter 43, and superimpose the DC signal Sdc of B volt (V) as shown in FIG. 8B onto the corresponding AC signal Sac′, thereby generating a final driving signal Vd as shown in FIG. 8C.

In this case, the voltage value of the DC signal Sdc may be set to be equal to or greater than the amplitude of the AC signal Sac′. Accordingly, the voltage value of the driving signal Vd may be set to zero or more.

As a result, the DC offset value of the driving signal Vd may be set to be equal to or greater than the amplitude of the driving signal Vd.

When the DC offset value of the driving signal Vd is less than the amplitude value of the driving signal Vd, a section occurs in which the voltage of the driving signal Vd has a negative value, and a loss of electrical energy occurs as the voltage becomes negative in the above section.

However, as in an embodiment of the present invention, if the DC offset value of the driving signal Vd is set to be equal to or greater than the amplitude of the driving signal Vd, the voltage of the driving signal Vd is always equal to or greater than zero, so the loss of electrical energy may be minimized.

Meanwhile, the DC signal Sdc may be generated by the voltage divider 45. For example, the voltage divider 45 may receive the output voltage Vo of the DC-DC converter 41, and may perform voltage division on the output voltage Vo to generate the DC signal Sdc.

The voltage divider 45 may be composed of a resistor string for dividing the output voltage Vo, but is not limited thereto.

When the output voltage Vo of the DC-DC converter 41 is suitable for direct use in generating the driving signal Vd, the corresponding output voltage Vo may serve as the DC signal Sdc. In this case, the voltage divider 45 may be omitted, and the output voltage Vo of the DC-DC converter 41 may be inputted to the calibrator 44.

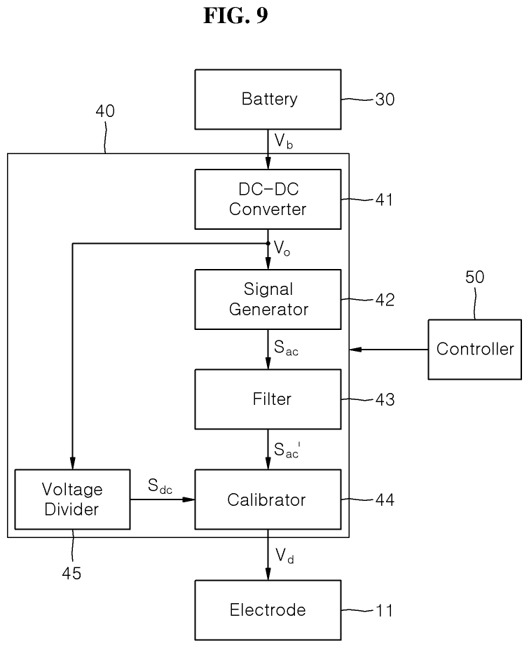

FIG. 9 is a diagram showing an internal configuration of the skin inflammation treatment device according to another embodiment of the present invention.

Referring to FIG. 9, in the skin inflammation treatment device 1 according to another embodiment of the present invention, the signal supply 40 may change at least one characteristic of the driving signal Vd under the control of the user. In addition, the skin inflammation treatment device 1 may additionally include the controller 50 that controls the signal supply 40 in response to the user's input.

For example, the characteristics of the driving signal Vd may include the amplitude and DC offset of the driving signal Vd.

Accordingly, the user may set an optimal driving signal Vd suitable for his/her use by adjusting at least one of the amplitude and DC offset of the driving signal Vd.

In this case, the user's input method for controlling the characteristics of the driving signal Vd may be through the switch 23 or a separate button (not shown) installed on the skin inflammation treatment device 1, and the user may adjust or set the characteristics of the driving signal Vd by manipulating the switch 23 or the like.

When setting information of the driving signal Vd is inputted through a switch manipulation by the user, the controller 50 may control the signal supply 40 to provide the driving signal Vd having amplitude and DC offset values corresponding to the inputted setting information.

The controller 50 may change the amplitude of the AC signal Sac by controlling the signal generator 42. Further, the controller 50 may adjust the voltage value of the DC signal Sdc by controlling the DC-DC converter 41 and/or the voltage divider 45. Accordingly, the characteristics of the driving signal Vd may ultimately be changed.

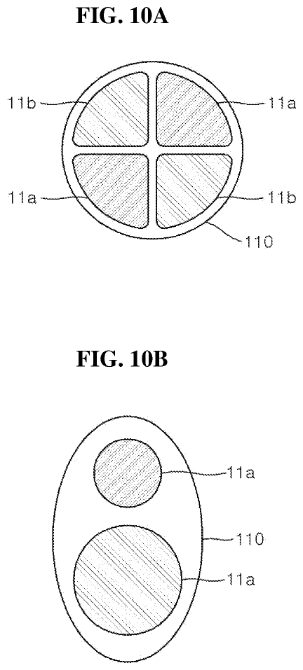

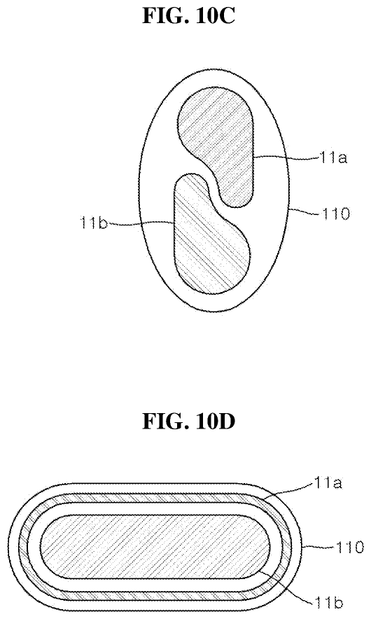

In this case, the controller 50 may control the voltage divider 45 to set the voltage value of the DC signal Sdc to be equal to or greater than the amplitude of the AC signal Sac′. Therefore, the voltage value of the driving signal Vd may be set be equal to or greater than zero. FIGS. 10A to 10D are diagrams illustrating the shape and arrangement of electrodes according to another embodiment of the present invention.

FIG. 4 and the like described above show an embodiment in which one first electrode 11a and one second electrode 11b each having a semicircular shape are provided, but the shape, number, arrangement structure, and the like of the first and second electrodes 11a and 11b may be varied.

For example, as shown in FIG. 10A, two first electrodes 11a and two second electrodes 11b may be provided and arranged to be spaced apart from each other. In this case, each electrode may have the same shape and may be fan-shaped as shown in the drawing.

Referring to FIG. 10B, one first electrode 11a and one second electrode 11b each having a circular shape may be provided and arranged to be spaced apart from each other, and the sizes of the first electrode 11a and the second electrode 11b may be different from each other.

Referring to FIG. 10C, one first electrode 11a and one second electrode 11b each having a curved shape may be provided and arranged to be spaced apart from each other, and the shapes of the first electrode 11a and the second electrode 11b may have the same.

Finally, referring to FIG. 10D, one first electrode 11a and one second electrode 11b may be provided, wherein the second electrode 11b may be disposed in a central region, and the first electrode 11a, which has a band shape surrounding the second electrode 11b, may be disposed to be spaced apart from the second electrode 11b.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

1. A skin inflammation treatment device comprising:
a case;
at least one electrode located in the case and configured to receive a driving signal and provide electromagnetic waves corresponding to the driving signal; and
a signal supply located in the case and configured to generate the driving signal by mixing an AC signal and a DC signal, and to supply the driving signal to the electrode.

2. The skin inflammation treatment device of claim 1, wherein the case includes:
a first case having an opening that exposes at least a portion of the electrode; and
a second case provided with a switch controlling the operation of the skin inflammation treatment device.

3. The skin inflammation treatment device of claim 2, wherein the first case includes a protrusion region, and the opening is formed in the protrusion region.

4. The skin inflammation treatment device of claim 2, further comprising:
a first circuit board on which the at least one electrode is mounted on a front surface thereof;
a second circuit board connected to the first circuit board through a connection line and on which the signal supply is mounted; and
a battery configured to supply a battery voltage to the signal supply.

5. The skin inflammation treatment device of claim 4, wherein the at least one electrode includes a first electrode and a second electrode arranged to be spaced apart from each other, and
wherein:
the first electrode includes a first insertion protrusion formed to protrude from one surface of the first electrode,
the second electrode includes a second insertion protrusion formed to protrude from one surface of the second electrode, and
the first circuit board includes a first receiving groove configured to receive the first insertion protrusion and a second receiving groove configured to receive the second insertion protrusion.

6. The skin inflammation treatment device of claim 5, wherein the first circuit board includes:
a first connection terminal located on a rear surface of the first circuit board and electrically connected to the first electrode; and
a second connection terminal located on the rear surface and electrically connected to the second electrode.

7. The skin inflammation treatment device of claim 4, further comprising:
a first indicator provided on the second case and configured to indicate a charging status of the battery; and
a second indicator provided on the second case and configured to indicate an operation time of the skin inflammation treatment device.

8. The skin inflammation treatment device of claim 1, further comprising:
a controller configured to control the signal supply to change at least one of characteristics of the driving signal under a user's control.

9. The skin inflammation treatment device of claim 8, wherein the characteristics of the driving signal include amplitude and DC offset.
